(19) United States
(12) Patent Application Publication
(10) Pub. No.: US 2016/0228283 A1
(43) Pub. Date: Aug. 11, 2016

(54) OSTOMY POUCH LAMINATE

(71) Applicant: WILTON TRUSTEES (IOM) LIMITED, Isle of Man (GB)

(72) Inventor: Kim Patchett, Essex (GB)

(73) Assignee: WILTON TRUSTEES (IOM) LIMITED, Isle of Man (GB)

(21) Appl. No.: 15/029,636

(22) PCT Filed: Oct. 8, 2014
(86) PCT No.: PCT/GB2014/053033

§ 371 Date: Apr. 15, 2016

(30) Foreign Application Priority Data
Oct. 16, 2013 (GB) 1318340.5

(51) Int. Cl. A61F 5/445; A61F 5/441; A61L 28/00

(52) U.S. Cl. CPC A61F 5/445; A61L 28/0053; A61L 28/0015; A61L 28/0019; A61L 28/0092; A61F 5/441

(57) ABSTRACT

A laminate (200) for a pouch (100) for collecting excreted waste, such as an ostomy pouch, comprises one or more porous layers (5). An adsorbent material is impregnated within the one or more porous layers (5) and a first polymer sheet (8) is attached to a first side of the one or more porous layers (5). Gases can permeate into the one or more porous layers (5), allowing odorous gases to be filtered via adsorption by the adsorbent material that is within the one or more porous layers (5).

This invention relates to a laminate for a pouch for collecting excreted waste and a method of manufacturing a laminate for a pouch for collecting excreted waste. This invention relates particularly but not exclusively to laminates for ostomy pouches.

There are a number of commercially available laminate polymer films currently used in the manufacture of ostomy pouches. For example, typical commercially available laminates comprise of a layer or layers of Polyvinylidene Chloride (PVdC) in combination with other polymers or copolymers to create multiple layer films. PVdC exhibits excellent barrier resistance to water vapour, oxygen and odours. However, there are potential dangers to human health issues arising at the end of life disposal of films and/or laminates that contain PVdC.

In particular, PVdC is a carbon based plastic polymer that contains Chlorine atoms in its polymer structure. Hazards can arise from the uncontrolled burning of chlorine containing plastic polymers, which may lead to the formation of dioxins and furans, which are highly toxic. The United Nations Environment Programme document UNEP/POPS/EGB.2/INF.6 entitled Information and Comments Received on Open Burning of 23 Oct. 2003 contains these statements:

“PCDD (=dioxins)/PCDF (=furans) can form due to incomplete combustion of carbon in the presence of chlorine. Open or uncontrolled burns represent poor fuel/oxidant mixtures leading to un-combusted carbon. If chlorine is present, reactions with the carbon structures may lead to PCDD/PCDF formation. The practice of open combustion must ensure that the conditions for generating PCDD/F are minimized and eliminated”; and “Avoid mixed fuels with contaminants of chlorine or products made with chlorine”.

Other commercially available laminate polymer films currently used in the manufacture of ostomy pouches comprise Ethylene Vinyl Acetate (EVA) or Ethylene Vinyl Alcohol (EVOH) within the laminate structure. Both EVA and EVOH exhibit excellent barrier resistance to oxygen and odours. However, in the case of EVOH, this barrier property diminishes in conditions where the EVOH is exposed to relative humidity (RH) levels in excess of 55%.

In recent years, there have been considerable advances in the development and commercial availability of polymers from biological sources such as Thermoplastic Starch (TPS), Polyactic acid (PLA), Polyhydroxyalkanoate; Poly(hydroxybutyrate-co-hydroxyvalerate (PHBv), Polyhydroxyalkanoate (PHA), Polyhydroxybutyrate (PHB) and others. Other biodegradable polymers derived from fossil fuel sources include Polycaprolactone (PCL), Polyesteramide (PEA), Aliphatic co-polyester; Poly(butylene succinate adipate) (PBSA) and others.

Generally speaking, polymers derived from biological sources generate lower carbon dioxide output during both the production and the disposal phase of their useful lifecycle. A summarised overview of the lifecycle assessment of non-biodegradable fossil fuel derived polymers typically used in barrier laminates for ostomy products is shown below in Table 1:

TABLE 1

| | | | | Greenhouse | |
|---|---|---|---|---|---|
| | | | | Gas | |
| | | | Cradle to Gate | Emissions | |
| Type of | | | energy use | (disposal) | |
| Polymer | Source | Unit | MJ/Kg (Kw*h) | Kg•CO₂/Kg | Reference |
| PVdC | Fossil Fuel | Kg | 80.6 (22.48) | 5.47 | Plastics Europe |
| | | | | | Eco Profile 2009 |
| Nylon MXD-6 | Fossil Fuel | Kg | 120 (33.33) | 7.64 | Bousted 1999 |
| PAN | Fossil Fuel | Kg | 108 (30.02) | 7.24 | EC-ELCD II Core |
| Polyacronynitrile | | | | | Data Set |
| LDPE | Fossil Fuel | Kg | 91.7 (25.47) | 5.20 | Dinkel et al 1999 |
| HDPE | Fossil Fuel | Kg | 79.9 (22.29) | 4.84 | Bousted 1999 |
| EVA | Fossil Fuel | Kg | 81.7 (22.75) | 5.83 | Bousted 1999 |
| Ethylene vinyl | | | | | |
| acetate | | | | | |

A summarised overview of the lifecycle assessment of some biodegradable and biodegradable/fossil fuel derived polymers is shown below in Table 2:

TABLE 2

| | | | Cradle | Greenhouse | |
|---|---|---|---|---|---|
| | | | to Gate | Gas | |
| | | | Energy use | Emissions | |
| Type of | | | MJ/Kg | (disposal) | |
| Polymer | Source | Unit | (Kw*h) | Kg•CO₂/Kg | Reference |
| TPS | Biopolymer | Kg | 25.4 (7.05) | 1.02 | Patel et al |
| | | | | | 1999 |
| TPS/PVOH | 85% | Kg | 24.9 (6.91) | 1.7 | Patel et al |
| | Biopolymer | | | | 1999 |

Biodegradable polymers may be derived from biological and non-biological sources. A summarised overview of the types and sources of a range of biodegradable polymers is shown below in Table 3:

TABLE 3
Biodegradable Polymers

| | From biotechnology: | From petrochemical |
|---|---|---|
| From micro-organisms: | (synthesis from bio | products: synthesis from |
| (obtained by extraction) | derived monomers) | synthetic monomers |
| Polyhydroxyalkanoates | Polyactides | Polycaprolactones |
| (PHA) | | (PCL) |
| Polyhydroxybutyrate | Poly(lactic acid) | Polyesteramides |
| (PHB) | (PLA) | (PEA) |
| Poly(hydroxybutyrate | | Aliphatic co-polyesters |
| co-hydroxyvalerate) | | (e.g. PBSA) |
| (PHBv) | | |
| | | Aromatic co-polyesters |
| | | (e.g. PBAT) |
| From biomass products from | | |
| agricultural resources (agro-polymers) | | |
| Polysaccharides | Proteins, Lipids | |
| Starches | Animals | |
| Wheat, Maize etc. | Whey, Gelatine etc. | |
| Ligno-cellulosic | Plant | |
| Wood, Straw etc. | Soya, Gluten etc. | |
| Others | | |
| Pectin, Gums etc. | | |

Film laminates used in the manufacture of ostomy pouches are normally required to have low moisture vapour transmission rates and low permeability to gases, vapours and odours.

A summary of the sulphur containing odorous compounds in human faeces is summarised in Table 4.

TABLE 4

| | | Concentration |
|---|---|---|
| Type of Gas | Gas | (parts per billion) |
| Sulphur Containing | Hydrogen Sulphide | 5-26 |
| | Methyl Mercapatan | 2-15 |
| | Methyl Sulphide | Not Detected |
| | Dimethyl Sulphide | Not Detected |

(Source: Sato et al, 2002)

A summary of the percentage of gas in total human flatus passed in a 4-hour period is summarised in Table 5.

TABLE 5

| Type of Gas | Gas | Percentage of Total |
|---|---|---|
| Non-odorous | Hydrogen | 34.3 ± 17.5 |
| | Carbon Dioxide | 34.7 ± 14.7 |
| | Methane | 5.6 ± 10.4 |
| | Oxygen | 3.3 ± 1.9 |
| | Nitrogen | 22.2 ± 12.2 |
| Sulphur Containing | Hydrogen Sulphide | 2.9 ± 4.0 (×10⁻³) |
| | Methyl Mercapatan | 0.58 ± 0.67 (×10⁻³) |
| | Dimethyl sulphide | 0.19 ± 0.2 (×10⁻³) |

(Source: Suarez et al, 1997)

In particular, film laminates used in the manufacture of ostomy pouches are required to have high resistance to the transmission of odours from compounds exuded from the human digestive system such as may arise from, for example, hydrogen sulphide and methyl mercapatan.

However, polymers derived from biological sources and biodegradable polymers can have relatively high moisture vapour transmission rates and relatively high permeability to gasses, vapours and odours. Thus, it has previously not been feasible to manufacture ostomy pouches from polymers derived from biological sources and biodegradable polymers.

A further issue with all current polymer films used for the manufacture of ostomy pouches is one of noise. It is important for the users of ostomy pouches to maintain discretion that they are wearing an ostomy pouch, and consequently it would be desirable for the laminate from which the pouch is made to be soft and quiet when crumpled or folded whilst being worn by the user to accommodate the natural movement or motion by the user in normal day to day activities. Unfortunately, it is a characteristic of all current commercially available polymer films for use in ostomy pouch manufacture that they are not particularly soft and quiet and are relatively noisy when crumpled or folded.

It is therefore desirable to provide a laminate suitable for use in ostomy pouch manufacture that can be made from a wider variety of materials, for example such that the laminate and/or pouch need not contain chlorine, need not contain vinyl, and may be produced substantially from bio-sources and/or biodegradable materials, but still has excellent odour control properties. It is also desirable to provide a laminate that is quieter when crumpled or folded.

Thus, according to the present invention there is provided a laminate for a pouch for collecting excreted waste, for example an ostomy pouch, the laminate comprising one or more porous layers, an adsorbent material being impregnated within the one or more porous layers, and a first polymer sheet attached to a first side of the one or more porous layers.

It will be understood that the one or more porous layers, being porous in nature, do not in themselves act as a barrier to gases. Instead, gases can permeate into the one or more porous layers. However, rather than simply passing straight through the one or more porous layers, odorous gases are filtered via adsorption by the adsorbent material that is within the one or more porous layers. Since the one or more porous layers and other layers of the laminate do not need to act as an absolute barrier to all gases, the laminate may be formed from a wider variety of materials. This means, for example, that the laminate does not need to contain chlorine or vinyl, and/or may be produced substantially from bio-sources and/or biodegradable materials, if desired. Furthermore, it has been found that the use of the one or more porous layers in the laminate can have a dramatic quieting effect on any polymer sheets that form part of the laminate. This is the case even for polymer sheets that comprise polymers conventionally used for ostomy pouches such as PVdC, EVA or EVOH.

The one or more porous layers preferably comprise one or more of: non-woven fabric; woven fabric; paper; felt; and open cell foam. Although the one or more porous layers may comprise non bio-sourced and/or non-biodegradable materials, in preferred embodiments the one or more porous layers comprise (only) bio-sourced and/or biodegradable material. The bio-sourced and/or biodegradable material of the one or more porous layers may comprise a polymer obtained by extraction from micro-organism, for example one or more of: Polyhydroxyalkanote (PHA), Polyhydroxybutyrate (PHB); and Poly(hydroxybutyrate co-hydroxyvalerate) (PHBv). Alternatively, the bio-sourced and/or biodegradable material of the one or more porous layers may comprise material obtained by synthesis from bio-derived monomers, for example one or more of: Polyactides; and Poly(lactic acid) (PLA). Alternatively, the bio-sourced and/or biodegradable material of the one or more porous layers may comprise material derived from agricultural resources, for example one or more of: starch based fibres; cellulose based fibres; Ligno-cellulosic based polymers; cotton fibres; pectin; and gum.

In preferred embodiments, the adsorbent material comprises molecular sieve material. It will be understood that molecular sieve materials are specifically not masking agents or additives with lower odour levels. The incorporation of molecular sieve materials into the aforementioned laminate structure results in entrapping odorous molecules from the immediate surrounding environment rather than creating a physical barrier to those odours as would be the case with a laminate containing chlorine or vinyl components.

A mechanism of adsorption of water molecules by a molecular sieve may be due to water molecules being polar (positively charged) therefore they are attracted to the structure of the molecular sieve which may inherently carry a negative charge.

When molecular sieves are incorporated into a laminate structure to create a composite they can act as an adsorbent to the transmission of a range of molecules that have a critical diameter smaller than the pore size of the internal cavities of the molecular sieve. This property has the effect of the porous layer, impregnated with molecular sieve material, replicating a barrier, by adsorption, to the passage of molecules of smaller critical diameter than the diameter of the pore size of the incorporated molecular sieve material and preventing or significantly reducing the rate of transmission of molecules through the laminate.

Odorous compounds that have been identified as constituent in human faeces and or human flatus and or human urine are shown in Table 6 below;

TABLE 6

| | | Critical Molecular |
|---|---|---|
| Type of Gas or Vapour | Gas or Vapour | Diameter (Å) |
| Non-Odorous | Hydrogen | 2.4 Å |
| | Carbon dioxide | 2.8 Å |
| | Methane | 4.0 Å |
| | Oxygen | 2.8 Å |
| | Nitrogen | 3.0 Å |
| | Water | 3.2 Å |
| Odorous | 3-methylindole | 7.9 Å |
| | Hydrogen sulphide | 3.6 Å |
| | Methyl mercapatan | 4.5 Å |
| | Dimethyl sulphide | 4.2 Å |
| | Ammonia | 3.6 Å |

For example, a hydrogen sulphide molecule has a critical molecular diameter of 3.6 Å and would be adsorbed by a molecular sieve with a pore size of approximately 4 Å or greater. Thus, the internal cavities of the molecular sieve material preferably have a nominal pore diameter in the range of 3 Å to 10 Å, and/or of approximately 4 Å or greater.

The adsorbent material may comprise one or more of: zeolites; faujasite; porous glass; activated carbon; clay; montmorillonite; halloysite; silicon dioxide; mesoporous silica; and sodium alumino-silicate.

The rate at which molecules of compounds are able to pass through the composite laminate sheet may be dependent upon the amount of adsorbent material contained within the one or more porous layers. In preferred embodiments, the one or more porous layers comprise a ratio of adsorbent material to porous material in the range 25-200%, 50%-150% or 75%-125%, or of approximately 100%, by weight.

Similarly, in a case where 100% of the voids in the porous layer are filled with impregnated adsorbent material, the rate at which compounds are able to pass through the laminate structure would be very low. In a case where 1% of the voids in the porous layer are filled with impregnated adsorbent material, the rate at which compounds are able to pass through the laminate structure would be relatively high. In embodiments, 1%-100% of the voids in the porous layer are filled with adsorbent material, preferably 20-80% of the voids in the porous layer are filled with adsorbent material, and more preferably 40-60% of the voids in the porous layer are filled with adsorbent material, for example approximately 50% of the voids in the porous layer are filled with adsorbent material.

The laminate preferably further comprises a second polymer sheet attached to a second side of the one or more porous layers. In some embodiments, the first and second polymer sheets each comprise first openings therethrough, such that the one or more porous layers provide a filter medium between the first openings. These embodiments allow, for example, flatus gas to enter the laminate via the first opening of the first polymer layer, to be filtered by the one or more porous layers, and to exit via the first opening of the second polymer layer. Thus, a build-up of flatus gas within a pouch having the laminate may be prevented, whilst malodours are still filtered out.

In preferred versions of these embodiments, the first opening of the first polymer sheet is not opposite the first opening of the second polymer sheet, and the first openings are bounded by a closed path along which the first polymer sheet is joined or fused to the second polymer sheet, thereby defining a filter path between the first openings that is bounded by the first and second polymer sheets through the one or more porous layers. The filter path may be elongate, curved, and/or spiral shaped. This can provide a defined and/or longer filter path for a given surface area of the laminate.

The first and second polymer sheets may each further comprise second openings therethrough, the second opening of the first polymer sheet being opposite the first opening of the second polymer sheet, the second opening of the second polymer sheet being opposite the first opening of the first polymer sheet. This feature may result from using a convenient method for forming the filter in which the opposite openings are formed by providing (e.g. punching or cutting) holes through the laminate. The first openings are preferably each covered by a liquid barrier and/or gas permeable membrane, and/or the second openings are preferably each covered by a gas impermeable cover.

In preferred embodiments, the or each polymer sheet is attached to the side of the one or more porous layers with an inter-laminar adhesive or by extrusion coating or by heat lamination. Although the or each polymer sheet may comprise non bio-sourced and/or non-biodegradable materials, in preferred embodiments the or each polymer sheet comprises bio-sourced and/or biodegradable material. The bio-sourced and/or biodegradable material of the or each polymer sheet may comprise a polymer obtained by extraction from micro-organism, for example one or more of: Polyhydroxyalkanote (PHA), Polyhydroxybutyrate (PHB); and Poly(hydroxybutyrate co-hydroxyvalerate) (PHBv). Alternatively, the bio-sourced and/or biodegradable material of the or each polymer sheet may comprise material obtained by synthesis from bio-derived monomers, for example one or more of: Polyactides; and Poly(lactic acid) (PLA). Alternatively, the bio-sourced and/or biodegradable material of the or each polymer sheet may comprise material derived from agricultural resources, for example one or more of: starch based fibres; cellulose based fibres; Ligno-cellulosic based polymers; cotton fibres; pectin; and gum.

As discussed above, the porous layer(s), polymer sheet(s) and/or laminate need not contain chlorine and/or vinyl. Thus, in preferred embodiments, the porous layer(s), polymer sheet(s) and/or laminate contains lower amounts of chlorine and/or vinyl than conventional laminates, and may for example not contain any chlorine and/or vinyl at all.

In preferred embodiments, the one or more porous layers comprise two, three or more porous layers. The two, three or more porous layers are preferably attached to one another with an inter-laminar adhesive or by extrusion coating or by heat lamination. These additional layers can increase structural integrity and/or reduce the transmission rate of malodours.

The present invention also extends to a pouch for collecting excreted waste, for example an ostomy pouch, the pouch comprising one or more laminates as described above.

The pouch preferably has a waste reservoir formed from the laminate sheet of the present invention. The pouch for collecting waste excreted from the human body, for example an ostomy pouch, may be substantially biodegradable and compostable and therefore provides lower carbon di-oxide emissions both in the manufacturing and disposal phases of the product lifecycle.

The bodily waste may be excreted via a stoma in the body wall; the stoma may be natural, or surgically created. Thus, the bodily waste may be, for example, urine, faeces, material diverted from the duodenum, jejunum, ileum, cecum, appendix, colon, rectum or other region of the alimentary canal, or material diverted from the gall bladder or bile duct. For the purposes of the invention, such bodily waste is considered ‘fluid’ and, therefore, suitable for collection, transportation and storage by devices described as suitable for use with fluids.

The pouch may be adapted for the collection of waste excreted via a stoma. The reservoir may have a fluid inlet connectable to a collector adapted for the collection of bodily waste excreted from the body. Alternatively, the fluid inlet may be connected to the collector. The collector may be, for example, a sheath attached to the penis.

In some embodiments the reservoir may have a fluid inlet connectable to a conduit, wherein the conduit is adapted to direct the excretion of bodily waste from the inside to the outside of the body. Alternatively, the fluid inlet may be connected to the conduit. The conduit may be, for example, a Foley or condom catheter.

The present invention also extends to a method of manufacturing a laminate, for example as described above, the method comprising impregnating an adsorbent material into one or more porous layers, and attaching a first polymer sheet to a first side of the one or more porous layers.

The method may comprise attaching a second polymer sheet to a second side of the one or more porous layers. As discussed above, the or each polymer sheet may be attached to a side of the one or more porous layers with an inter-laminar adhesive or by extrusion coating or by heat lamination.

The method may also comprise forming (e.g. punching or cutting) first and second holes into, or more preferably through, the laminate. The method may also then comprise joining or fusing the first polymer sheet to the second polymer sheet, the holes being bounded by a closed path along which the first polymer sheet is joined or fused to the second polymer sheet, thereby defining a filter path between the holes bounded by the first and second polymer sheets. The method may comprise covering first non-opposite openings of the holes each with a liquid barrier and/or gas permeable membrane, and/or covering second non-opposite openings of the holes each with a gas impermeable cover.

As discussed above, the one or more porous layers may comprise two, three or more porous layers, and the method may comprise attaching the two, three or more porous layers to one another with an inter-laminar adhesive or by extrusion coating or by heat lamination.

The present invention also extends to a method of manufacturing a pouch for collecting excreted waste, for example an ostomy pouch, the method comprising forming one or more walls of the pouch from one or more laminates as discussed above, or from one or more laminates manufactured using a method as discussed above.

The term ‘fluid’ used herein may include bodily waste as described herein and may also contain some solid material.

The porous layer(s), polymer sheet(s) and/or laminate are preferably ‘biodegradable’ or tompostable′. The terms ‘biodegradable’ and tompostable′ may refer to materials that meet the requirements of the International Standardisation Organisation standard EN 13432 Requirements for packaging recoverable through composting and biodegradation. Test scheme and evaluation criteria for the final acceptance of packaging.

The one or more porous layers may each have an areal weight of between 10 grams per square meter (gsm) and 500 gsm for example 20 gsm to 100 gsm or 25 gsm to 50 gsm. In one embodiment, the porous layers each have an areal weight of 40 gsm.

It has been found by experimentation that the period of time for which the laminate sheet remains an effective barrier by adsorption to odours and gases may be varied by modifying the areal weight of the porous layer(s) and the amount of adsorbent material incorporated into the porous layer(s).

For example, in one embodiment, a cellulosic non-woven fabric of areal weight of 40 gsm was impregnated with a Zeolite molecular sieve of pore diameter of 10 Å at 16 gsm, thereby providing a ratio of molecular sieve material to non-woven fabric of 40% by weight. On to each surface of the resulting composite was laminated a polymer sheet film by means of an adhesive interlayer and it was established that the resulting composite laminate structure prevented substantial odour and gas transmission by the composite laminate as a whole for at least 4 hours (Example 1).

In another embodiment, a cellulosic non-woven fabric of areal weight of 40 gsm was impregnated with a Zeolite molecular sieve of pore diameter of 10 Å at 24 gsm, thereby providing a ratio of molecular sieve material to non-woven fabric of 60% by weight. On to each surface of the resulting composite was laminated a polymer sheet film by means of an adhesive interlayer and it was established that the resulting composite laminate structure prevented substantial odour and gas transmission by the composite laminate as a whole for at least 8 hours (Example 2).

In a further embodiment, a cellulosic, bleached kraft paper of areal weight of 70 gsm was impregnated with a Zeolite molecular sieve of pore diameter of 10 Å at 28 gsm, thereby providing a ratio of molecular sieve material to non-woven fabric of 40% by weight. On to each surface of the resulting composite was laminated a polymer sheet film by means of an adhesive interlayer and it was established that the resulting composite laminate structure prevented substantial odour and gas transmission by the composite laminate as a whole for at least 10 hours (Example 3).

For a given porous layer and adsorbent material composite with a polymer film sheet attached to both surfaces to create a composite laminate, the time period for which the composite laminate as a whole acts as an effective barrier to odours and gas is proportional to the thickness of each component of the laminate, the ratio of adsorbent material to porous material and the ambient conditions of temperature and humidity in which the composite laminate resides. Accordingly, given this teaching, the skilled person can now readily adjust the thickness and construction of each of the laminate components to suit a specific application or performance requirement.

The laminate sheet of the present invention may incorporate one or more further layers in order to improve or modify particular barrier properties as required. In one embodiment, one or more further porous layer and adsorbent material composites are located adjacent to the porous layer and adsorbent material composite. The further layer(s) may incorporate a similar or different grade or type of adsorbent material or molecular sieve material to that of the first layer.

In a further embodiment, an additional layer or layers of polymer film sheet are located on at least one of the first and/or second polymer film sheets.

For example, if greater mechanical strength or further improved odour barrier properties are required for a particular application of the resulting laminate sheet, a further layer of polymer film sheet may be included in the laminate sheet. The further layer may be located at or close to the periphery of the laminate sheet and may be added to the primary layer or layers by, for example but limited to, heat-welding, extrusion coating, an adhesive laminating process or co-extruded in a single manufacturing process with the underlying layers.

A further layer may be included in the composite laminate structure for the purpose of absorbing sweat excreted by the wearer's body. This layer may be a layer of non-woven fabric. Preferably the layer is a non-woven, biodegradable and compostable fabric.

In one embodiment, the further sweat-absorbing layer is located adjacent to the outer polymer film sheet layer.

The laminate sheet may be used in the construction of a variety of biodegradable and compostable devices used for the collection of bodily waste.

As discussed above, it has been found that the use of the porous layer in the laminate can have a dramatic quieting effect on any polymer sheets within the laminate. Indeed, even laminates having barrier polymer film sheets conventionally used in ostomy bags can benefit from this feature.

Thus, according to another aspect of the present invention, there is provided a laminate for a pouch for collecting excreted waste, for example an ostomy pouch, the laminate comprising one or more porous layers and one or more polymer layers.

This laminate may comprise one or more of the above described preferred and/or optional features of the invention. For example, the one or more porous layers preferably comprise one or more of: non-woven fabric; woven fabric; paper; felt; and open cell foam. However, in this aspect, the one or more porous layers may not comprise an adsorbent material. The polymer layers may also comprise Polyvinylidene Chloride (PVdC), Ethylene Vinyl Acetate (EVA), and/or Ethylene Vinyl Alcohol (EVOH).

The invention also extends to a pouch having this laminate, a method of manufacturing this laminate by laminating one or more porous layers and one or more polymer layers, and a method of manufacturing a pouch having this laminate by forming the walls of the pouch from this laminate.

By way of example only, specific embodiments of this invention will now be described in detail with reference being made to the accompanying drawings in which:—

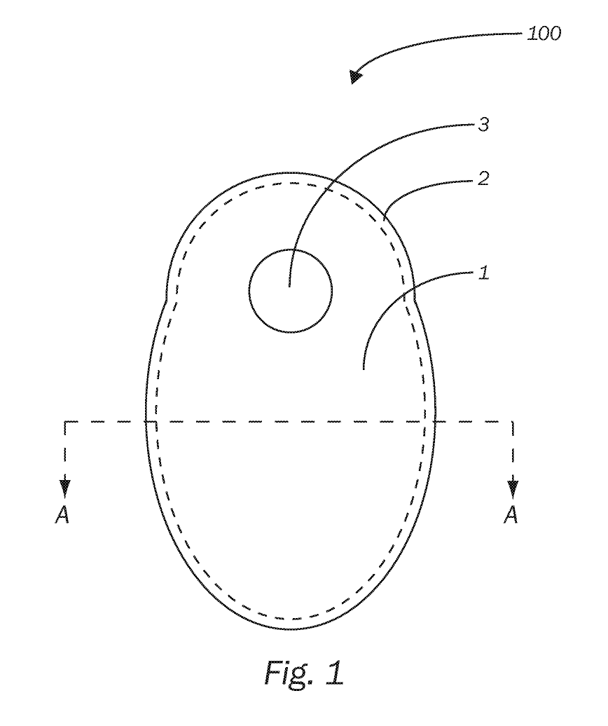

FIG. 1 shows an ostomy pouch having a laminate according to an embodiment of the invention.

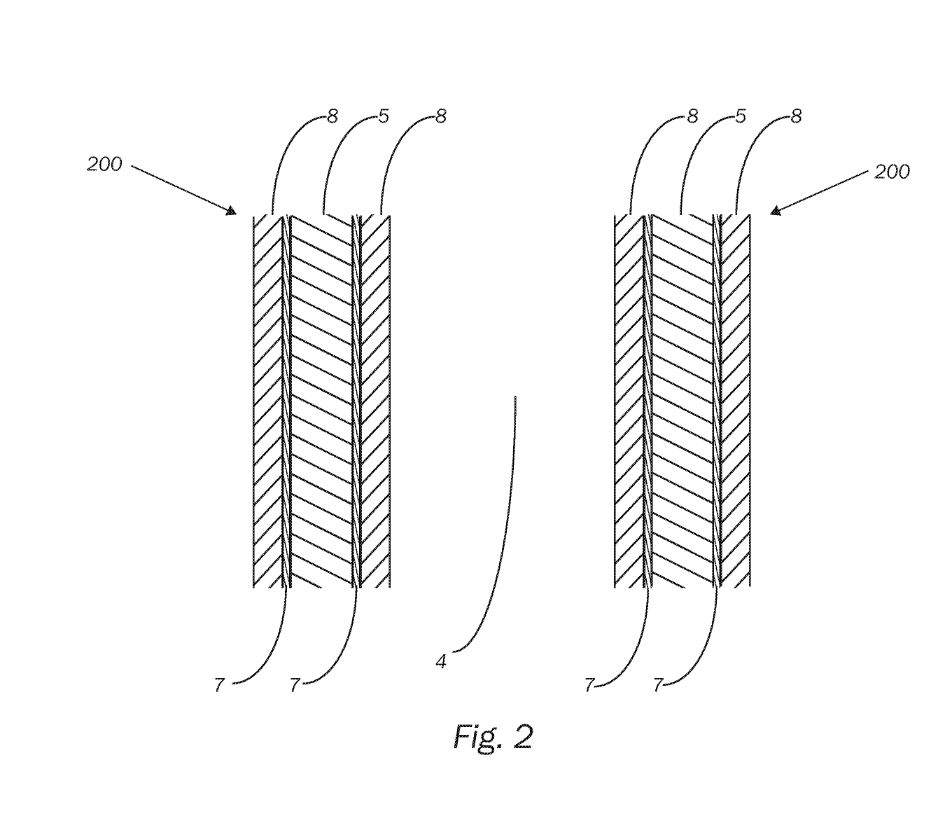

FIG. 2 shows a cross section through the laminate of the ostomy pouch of FIG. 1 according to one embodiment of the invention.

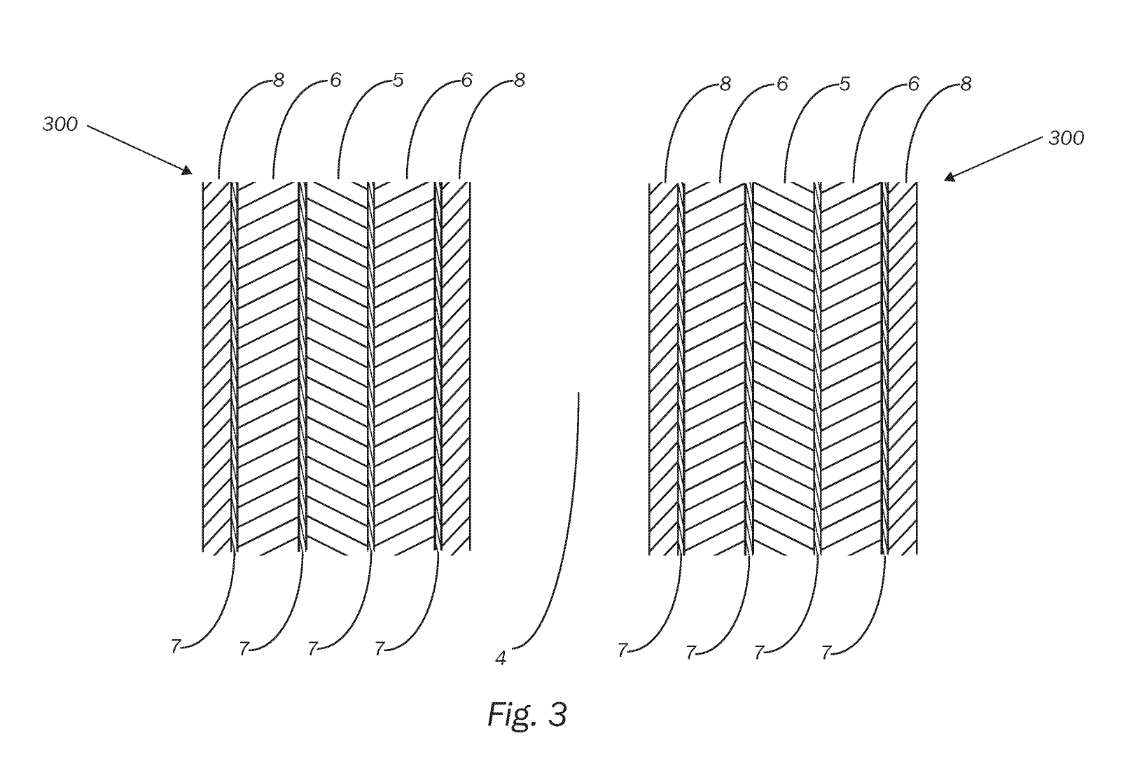

FIG. 3 shows a cross section through the laminate of the ostomy pouch of FIG. 1 according to another embodiment of the invention.

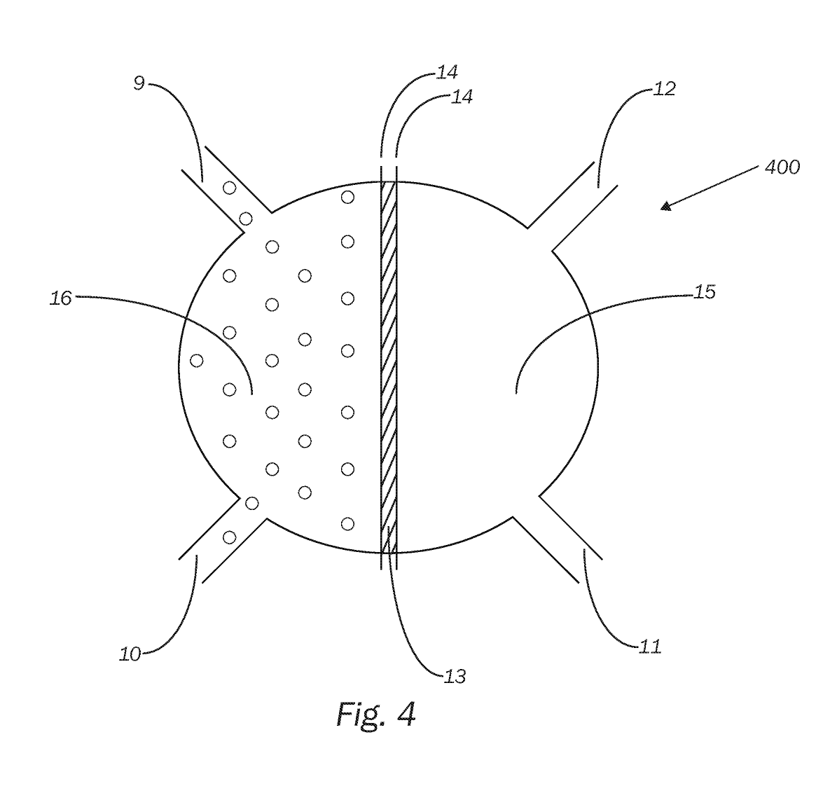

FIG. 4 shows a schematic diagram of a test apparatus used to detect the penetration of odours through a laminate.

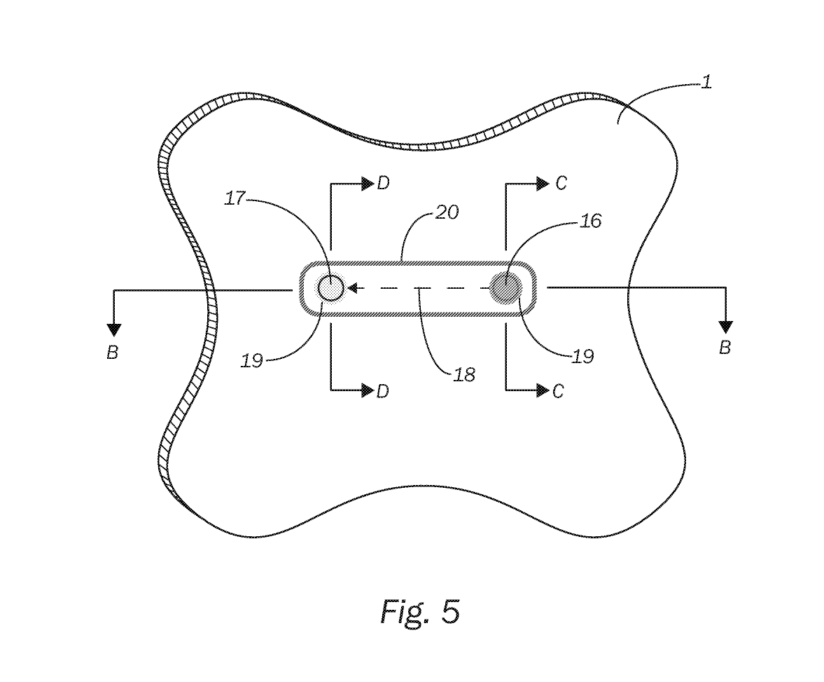

FIG. 5 shows a laminate having a filter path according to one embodiment of the invention.

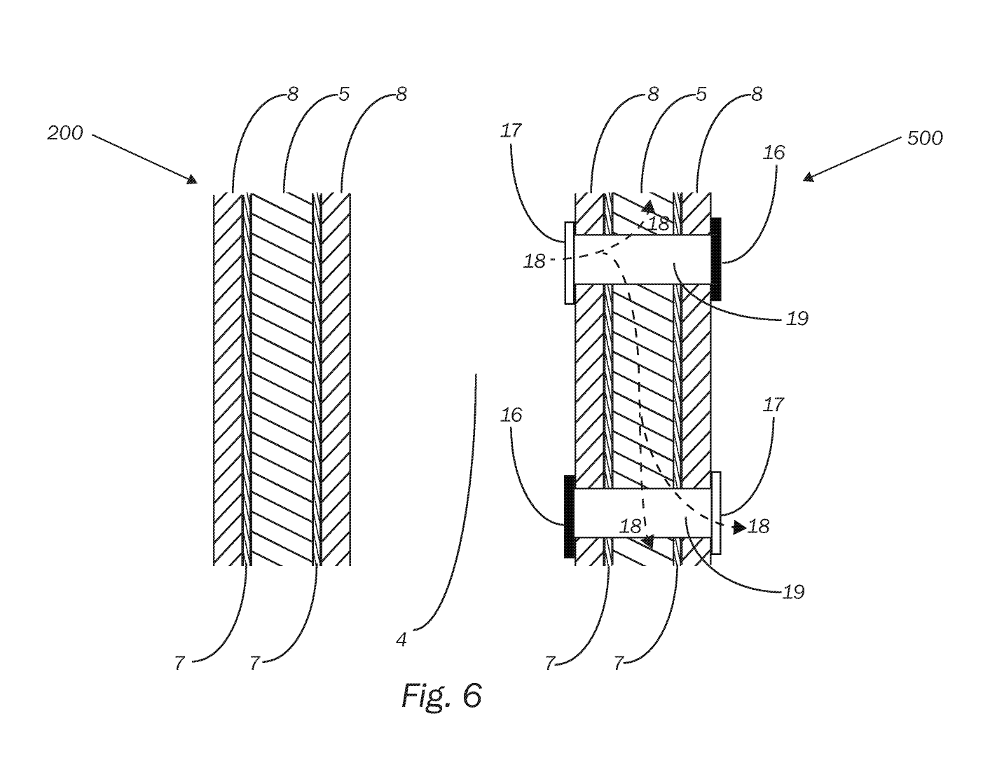

FIG. 6 shows a first cross section through the laminate of the ostomy pouch of FIG. 5.

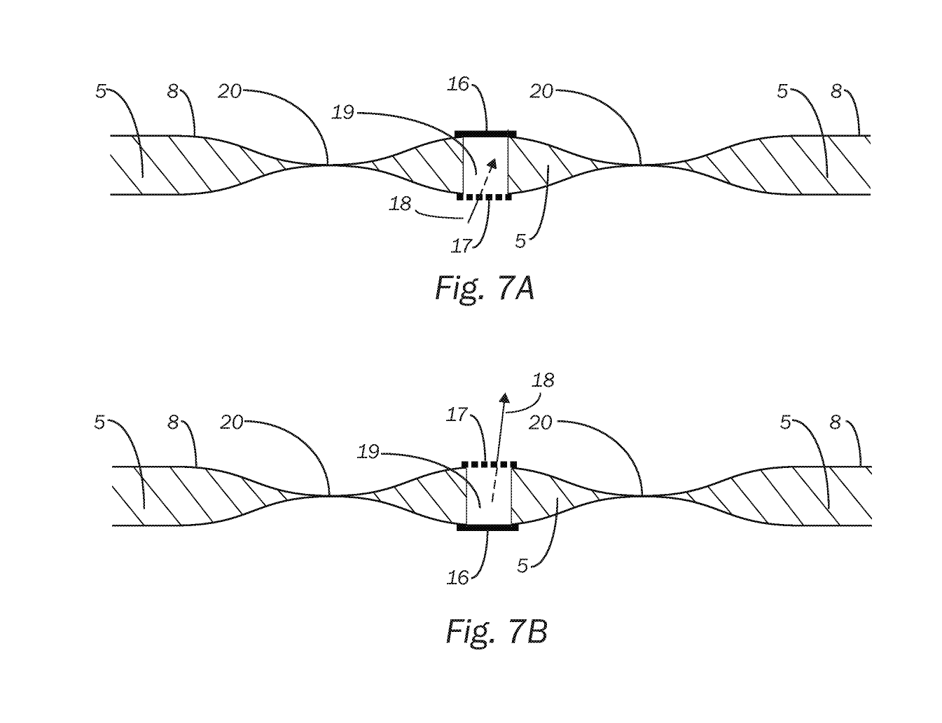

FIGS. 7A and 7B show second and third cross sections through the laminate of the ostomy pouch of FIG. 5.

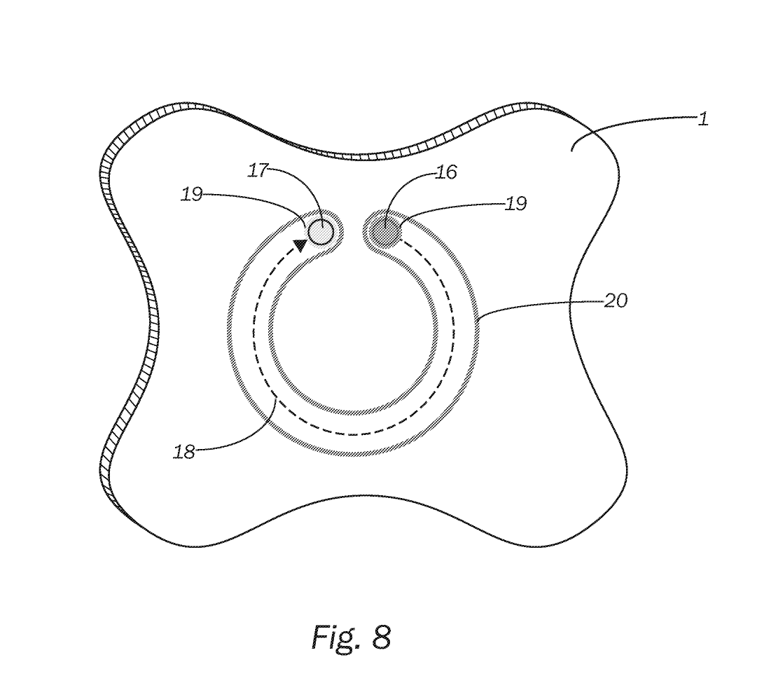

FIG. 8 shows a laminate having a filter path according to another embodiment of the invention.

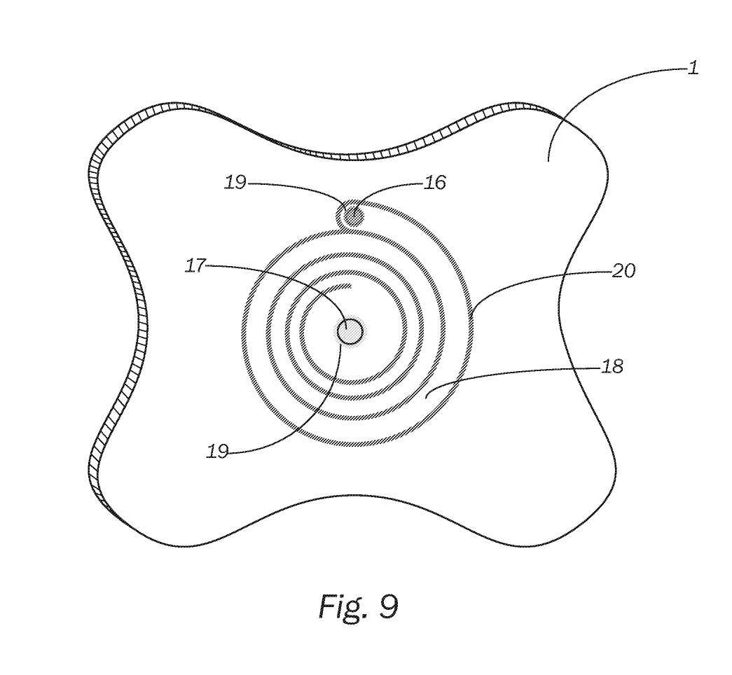

FIG. 9 shows a laminate having a filter path according to yet another embodiment of the invention.

FIG. 1 shows an ostomy pouch 100 in plan view with laminate walls 1 of the pouch 100 being joined by welding or bonding around the periphery 2 of the pouch 100 to form a receptacle device and with an opening 3 formed through one surface wall of the pouch 100 to allow waste products exuded from the user's stoma to pass into the pouch 100. It should be noted that a variety of joining devices may be utilised to join the pouch 100 to the user's abdomen.

FIG. 2 shows a cross section through FIG. 1 along A-A. FIG. 2 shows a pouch reservoir 4 for receiving and collecting waste products exuded from the user's stoma. The walls of the pouch 100 are constructed of a laminate 200 comprising a porous layer 5 having a biodegradable and compostable gas permeable porous structure and molecular sieve material, onto each surface of which is attached a layer of polymer film sheet 8 by means of an adhesive layer 7. In this embodiment, the biodegradable and compostable gas permeable porous structure and molecular sieve composite is of areal weight 40 gsm.

Two of the aforesaid five element composite laminate sheets 200 are then cut to the desired plan shape of the pouch 100 under construction and laid together, one sheet 200 directly on top of the other sheet such that the edges of both sheets 200 are accurately aligned when viewed in plan. The two laminate sheets 200 are then heat bonded or otherwise joined together at their periphery edge 2 to create the reservoir 4 and the opening 3 is created through one of the laminate sheets 200 to allow for the reception of waste products exuded from the stoma on the users abdomen. A connection device (not shown) is then attached to the pouch 100 within which has been formed the said opening 3 to allow connection of the whole assembly to the user's abdomen.

FIG. 3 shows a cross section through FIG. 1 along A-A according to another embodiment. FIG. 3 shows a pouch reservoir 4 for receiving and collecting waste products exuded from the user's stoma. The walls of the pouch 100 are constructed of a laminate 300 comprising a porous layer 5 having a biodegradable and compostable gas permeable porous structure and molecular sieve material, onto each surface of which is attached a further porous layer 6 having a biodegradable and compostable gas permeable porous structure and molecular sieve material by means of an adhesive layer 7. Two polymer film sheets 8 are attached to the outer surface of the porous layers 6 by means of an adhesive layer 7.

Two of the aforesaid nine element laminate sheets 300 are then cut to the desired plan shape of the pouch 100 under construction and laid together, one sheet 300 directly on top to the other sheet 300 such the edges of both sheets 300 are accurately aligned when viewed in plan. The two laminate sheets 300 are then heat bonded or otherwise joined together at their periphery edge 2 to create the reservoir 4 and the opening 3 is created through one of the laminate sheets to allow for the reception of waste products exuded from the stoma on the user's abdomen. A connection device (not shown) is then attached to the pouch 100 within which has been formed the said opening to allow connection of the whole assembly to the user's abdomen.

In order to illustrative the efficacy of the laminates of embodiments of the invention, a suitable testing procedure will now be described.

The testing procedure and standard requirements for ostomy pouch odour retention is set out by the International Standard Organisation standard ISO 8670-3:2000(E). The test procedure set out by this standard may be summarised as follows:

i) a moist odorous medium is placed within the test pouch and the opening of the pouch sealed;
ii) the sealed pouch is placed on a raised platform in an airtight container;
iii) distilled water is added to a level below that of the raised platform (so that the pouch is not directly in contact with the distilled water);
iv) the whole system is maintained at a temperature of 34±1° Celsius and at specified time intervals, the container is unsealed and any odour emanating from the interior of the container noted by olfactory means.

Whilst this standard is of merit in determining the odour barrier properties of a given film laminate, it can be somewhat subjective since the olfactory senses of given individuals performing the tests may differ.

Therefore, for the purposes of embodiments of this invention, an objective method of testing has been used in accordance with ASTM Standard F739-85 and the method of detection of odorous vapours or gasses passing through the laminate being tested measured by Fourier Transform Infrared (FT-IR) Spectroscopy thereby eliminating potential differences in human olfactory sensitivity. In embodiments, 3-methylindole in a solution of distilled water at the rate of 0.5 g per 1000 cc of distilled water was used as the challenge media for odour barrier testing at a test temperature of 34±1° Celsius and RH of 100%.

FIG. 4 shows a schematic diagram of a test apparatus 400 to detect the penetration of odours through the film laminate under test 13 that is secured in place in an airtight condition form the surrounding atmosphere by two Teflon™ gaskets 14. The challenge medium 16 enters into the test apparatus 400 through inlet 9 and exits the test apparatus 400 through outlet 10. On the collection side 15 of the apparatus, the collection medium enters the test apparatus 400 through inlet 11 and out of the collection side of the test apparatus 400 to the detector through outlet 12.

A detector measures the amount of challenge material that has permeated through the film laminate under test 13.

FIG. 5 shows a further embodiment of a laminate 1 for the ostomy pouch of FIG. 1 that prevents build-up of flatus gas whilst also filtering that gas. FIG. 6 shows a cross section through FIG. 5 along B-B. As can be seen from the figures, a pouch cavity 4 is provided for receiving and collecting waste products exuded from the user's stoma. The walls of the pouch 100 are constructed of laminates 200, 500 comprising a porous layer 5 having biodegradable and compostable gas permeable porous structure and molecular sieve material, onto each surface of which is attached a layer of polymer film sheet 8 by means of an adhesive layer 7. To alleviate the build-up flatus gas within the pouch, a liquid barrier membrane 17 is attached to the wall of the pouch over first openings of holes 19, which are cut or punched or otherwise formed in the pouch wall, by heat welding or with the use of an adhesive or other suitable means. On the opposite openings of the holes 19 is attached a gas impermeable cover 16 by heat welding or with the use of an adhesive or other suitable means, to create a filter path 18 for flatus gas to exit the pouch via the gas permeable porous structure and molecular sieve material of the porous layer 5 thereby removing any odours present in the flatus gas prior to venting to the outside of the pouch.

Referring again the FIG. 5, the filter path 18 is defined by a closed path 20 that bounds the holes 19. FIG. 7A shows a cross section through FIG. 5 along C-C and FIG. 7B shows a cross section through FIG. 5 along D-D. As can be seen from FIGS. 7A and 7B, the polymer film sheets 8 are joined or fused to one another along the closed path 20 so as to define the filter path 18 of the porous layer 5. The filter path 18 shown in FIG. 5 is elongate. This provides a long path over which the flatus gas can be filtered. Within a given surface area of the laminate, the filter path 18 can be made even longer by curving the filter path 18 (see FIG. 8), and made even longer still by spiralling the filter path 18 (see FIG. 9).

Several more specific exemplary embodiments will now be described.

### EXAMPLE 1

A 40 gsm layer of cellulosic non-woven fabric was impregnated with a Zeolite molecular sieve of pore diameter of 10 Å at 16 gsm, thereby providing a ratio by weight of molecular sieve material to non-woven fabric of 40%. On to each surface of the resulting composite was laminated a polyhydroxyalkanoate (PHA) polymer sheet film of 40 microns in thickness by means of an adhesive interlayer.

A sample of the resulting laminate was then tested in accordance with ASTM F 739-85 in conditions of 34±1° Celsius and at a relative humidity of 100% using a solution of 3-methylindole in solution with distilled water at a ratio of 0.5 g 3-methylindole to 1000 cc distilled water as the challenge media.

It was determined that the laminate described in example 1 prohibited the breakthrough of odours from the challenge media for a period of 4 hours.

### EXAMPLE 2

A 40 gsm cellulosic non-woven fabric of areal weight was impregnated with a Zeolite molecular sieve of pore diameter of 10 Å at 24 gsm, thereby providing a ratio by weight of molecular sieve material to non-woven fabric of 60%. On to each surface of the resulting composite was laminated a polyhydroxyalkanoate (PHA) polymer sheet film of 40 microns in thickness by means of an adhesive interlayer.

A sample of the resulting laminate was then tested in accordance with ASTM F 739-85 in conditions of 34±1° Celsius and at a relative humidity of 100% using a solution of 3-methylindole in solution with distilled water at a ratio of 0.5 g 3-methylindole to 1000 cc distilled water as the challenge media.

It was determined that the laminate described in example 2 prohibited the breakthrough of odours from the challenge media for a period of 8 hours.

### EXAMPLE 3

A cellulosic, bleached kraft paper of areal weight of 70 gsm was impregnated with a Zeolite molecular sieve of pore diameter of 10 Å at 28 gsm, thereby providing a ratio by weight of molecular sieve material to non-woven fabric of 40%. On to each surface of the resulting composite was laminated a polyhydroxyalkanoate (PHA) polymer sheet film of 40 microns in thickness by means of an adhesive interlayer.

A sample of the resulting laminate was then tested in accordance with ASTM F 739-85 in conditions of 34±1° Celsius and at a relative humidity of 100% using a solution of 3-methylindole in solution with distilled water at a ratio of 0.5 g 3-methylindole to 1000 cc distilled water as the challenge media.

It was determined that the laminate described in example 3 prohibited the breakthrough of odours from the challenge media for a period of 10 hours.

### EXAMPLE 4

A 50 gsm layer of cellulosic non-woven fabric was impregnated with a Zeolite molecular sieve of pore diameter of 10 Å at 25 gsm, thereby providing a ratio by weight of molecular sieve material to non-woven fabric of 50%. On to each surface of the resulting composite was laminated a biaxially oriented polypropylene polymer sheet film by means of an adhesive interlayer.

A sample of the resulting laminate was then tested in accordance with ASTM F 739-85 in conditions of 34±1° Celsius and at a relative humidity of 100% using a solution of 3-methylindole in solution with distilled water at a ratio of 0.5 g 3-methylindole to 1000 cc distilled water as the challenge media.

It was determined that the laminate described in example 4 prohibited the breakthrough of odours from the challenge media for a period of 25 hours.

### EXAMPLE 5

A 50 gsm layer of polyester non-woven fabric was impregnated with a Zeolite molecular sieve of pore diameter of 10 Å at 50 gsm, thereby providing a ratio by weight of molecular sieve material to non-woven fabric of 100%. On to each surface of the resulting composite was laminated a biaxially oriented polypropylene polymer sheet film by means of an adhesive interlayer.

A sample of the resulting laminate was then tested in accordance with ASTM F 739-85 in conditions of 34±1° Celsius and at a relative humidity of 100% using a solution of 3-methylindole in solution with distilled water at a ratio of 0.5 g 3-methylindole to 1000 cc distilled water as the challenge media.

It was determined that the laminate described in example 5 prohibited the breakthrough of odours from the challenge media for a period of 26 hours.

1. A laminate for a pouch for collecting excreted waste, the laminate comprising:
at least one porous layer, an adsorbent material being impregnated within the at least one porous layer; and
a first polymer sheet attached to a first side of the at least one porous layer.

2. A laminate as claimed in claim 1, further comprising a second polymer sheet attached to a second side of the at least one porous layer.

3. A laminate as claimed in claim 2, wherein the first polymer sheet and the second polymer sheet each comprise a first opening therethrough, the at least one porous layer thereby providing a filter medium between said first openings.

4. A laminate as claimed in claim 3, wherein the first opening of the first polymer sheet is not opposite the first opening of the second polymer sheet, said first openings each being bounded by a closed path along which the first polymer sheet is joined or fused to the second polymer sheet, thereby defining a filter path between said first openings, said filter path being bounded by said first and second polymer sheets.

5. (canceled)

6. A laminate as claimed in claim 3, wherein the first polymer sheet and the second polymer sheets each comprise a second opening therethrough, the second opening of the first polymer sheet being opposite the first opening of the second polymer sheet, and the second opening of the second polymer sheet being opposite the first opening of the first polymer sheet.

7. A laminate as claimed in claim 3, wherein said first openings are each covered by at least one of a liquid barrier and a gas permeable membrane.

8. A laminate as claimed in claim 1, wherein the at least one porous layer is gas permeable.

9. (canceled)

10. A laminate as claimed in claim 1, wherein at least one of: the at least one porous layer, and each said polymer sheet, comprises bio-sourced material.

11. A laminate as claimed in claim 8, wherein said bio-sourced material comprises at least one of:
a polymer obtained by extraction from micro-organism, said polymer comprising at least one of: Polyhydroxyalkanote (PHA), Polyhydroxybutyrate (PHB); and Poly(hydroxybutyrate co-hydroxyvalerate) (PHBv);
material obtained by synthesis from bio-derived monomers, said material comprising at least one of: Polyactides; and Poly(lactic acid) (PLA); and
material derived from agricultural resources, said material comprising at least one of: starch based fibres, cellulose based fibres, Ligno-cellulosic based polymers, cotton fibres, pectin, and gum.

12. A laminate as claimed in claim 1, wherein the adsorbent material comprises molecular sieve material, and wherein the internal cavities of the molecular sieve material have a nominal pore diameter in the range of 3 Å to 10 Å.

13. (canceled)

14. A laminate as claimed in claim 1, wherein the at least one porous layer comprising a ratio of adsorbent material to porous material in the range 25-200% by weight.

15. A laminate as claimed in claim 1, the adsorbent material comprising at least one of: zeolites, faujasite, porous glass, activated carbon, clay, montmorillonite, halloysite, silicon dioxide, mesoporous silica, and sodium alumino-silicate.

16. A laminate as claimed in claim 1, wherein each said polymer sheet is attached to a side of the at least one porous layer by one of: an inter-laminar adhesive, extrusion coating, and heat lamination.

17. (canceled)

18. (canceled)

19. A laminate as claimed in claim 1, wherein at least one of: the at least one porous layer, each said polymer sheet, and the laminate, do not contain at least one of chlorine and vinyl.

20. A laminate as claimed in claim 1, wherein the at least one porous layer comprises at least two porous layers.

21. A laminate as claimed in claim 20, wherein the at least two porous layers are attached to one another by one of: an inter-laminar adhesive, extrusion coating, and heat lamination.

22. A pouch for collecting excreted waste, said pouch comprising at least one laminate as claimed in claim 1.

23. A method of manufacturing a laminate as claimed in claim 1, the method comprising:
impregnating an adsorbent material into at least one porous layer; and
attaching a first polymer sheet to a first side of the at least one porous layer.

24. A method as claimed in claim 23, comprising attaching a second polymer sheet to a second side of the at least one porous layer.

25. A method as claimed in claim 24, comprising forming first and second holes in the laminate.

26-34. (canceled)
